(19) United States
(12) Patent Application Publication
(10) Pub. No.: US 2022/0401626 A1
(43) Pub. Date: Dec. 22, 2022

(54) LIQUID COMPOSITION AND POROUS HARDENED MATERIAL COMPRISING TETRAFLUOROETHYLENE AND VINYL MOIETY CO-POLYMERS

(71) Applicant: W. L. Gore & Associates, Inc., Newark, DE (US)

(72) Inventors: Paul D. Drumheller, Flagstaff, AZ (US); Aadil Elmoumni, Middletown, DE (US); Selim Ergen, Newark, DE (US); Eric A. Mokelke, Flagstaff, AZ (US); Mitchell Troutman, Flagstaff, AZ (US); Raquel Sanchez, Flagstaff, AZ (US)

(21) Appl. No.: 17/776,292

(22) PCT Filed: Nov. 13, 2020
(86) PCT No.: PCT/US2020/060419

§ 371 Date: May 12, 2022

Related U.S. Application Data
(60) Provisional application No. 62/934,650, filed on Nov. 13, 2019.

(51) Int. Cl. A61L 27/56; A61L 27/54; A61L 27/38; A61L 27/22; A61L 27/16

(52) U.S. Cl. CPC A61L 27/56; A61L 27/54; A61L 27/3804; A61L 27/22; A61L 27/16

(57) ABSTRACT

A porous hardened material is provided for various medical applications, including strengthening, supporting, moving, reinforcing, separating, isolating, and/or bulking biological substrates. The hardened material is formed from a liquid composition including a fluorinated copolymer and a biocompatible solvent system. The fluorinated copolymer includes a tetrafluoroethylene (TFE) moiety and a vinyl moiety, wherein the vinyl moiety comprises at least one functional group selected from acetate, alcohol, amine, and amide.

### CROSS REFERENCE TO RELATED APPLICATION

This application is a national phase application of PCT Application No. PCT/US2020/060419, internationally filed on Nov. 13, 2020, which claims priority to U.S. Provisional Patent Application Ser. No. 62/934,650, filed Nov. 13, 2019, the disclosures of which are hereby expressly incorporated by reference herein in their entireties.

### FIELD

The present disclosure relates to fluorinated copolymers, and more specifically, to fluorinated copolymers including a tetrafluoroethylene (TFE) moiety and a vinyl moiety for medical applications.

### BACKGROUND

TFE co-polymers are well known in the art. TFE co-polymers are of great use in many industries but are particularly useful in medical applications due to their inertness and biocompatibility.

While useful in many respects, utilizing TFE copolymers dissolved or otherwise prepared as a solution for medical applications poses difficulties. TFE copolymers that are water-soluble are not useful for many medical applications because they are not as inert or are resistant to dissolution in an aqueous environment. On the other hand, TFE copolymers that are insoluble in water or in biological fluids such as blood, serum, cerebrospinal fluid, interstitial fluid, and the like, are often very hydrophobic, which is also problematic. In particular, the solvents used to dissolve or otherwise solubilize these types of tetrafluoroethylene co-polymers may be unsuitable for in vivo use. Examples of said solvents include halogenated solvents, fluorinated kerosene solvents, aromatic solvents, and mineral acid solvents.

Porous hardened materials, such as gels and hydrogels comprising pores, are useful in medical applications due to their mechanical properties approximating the mechanical properties of biological substrates. These mechanical properties include rheological modulus, pore size, and pore distribution.

Therefore, there continues to be a need for creating TFE copolymers that are hydrophilic but not soluble in water, and that can be dissolved in a biocompatible solvent system such as a biocompatible solvent, a nonaqueous mixture of biocompatible solvents, or an aqueous mixture of biocompatible solvents. Additionally, there continues to be a need for creating liquid compositions comprising TFE copolymers that are hydrophilic but not soluble in water or a biological fluid. Additionally, there continues to be a need for creating TFE copolymers that are hydrophilic but not soluble in water, and that comprise porous hardened structures, without the limitations mentioned above.

### SUMMARY

A porous hardened material is provided for various medical applications, including strengthening, supporting, moving, separating, isolating, reinforcing, and/or bulking biological substrates. The hardened material is formed from a liquid composition including a fluorinated copolymer and a biocompatible solvent system comprising a biocompatible organic solvent, a mixture of biocompatible organic solvents, or an aqueous mixture of biocompatible organic solvents. The fluorinated copolymer includes a tetrafluoroethylene (TFE) moiety and a vinyl moiety, wherein the vinyl moiety comprises at least one functional group selected from acetate, alcohol, amine, and amide.

According to one example (“Example 1”), a porous material is disclosed including a plurality of filamentous structures including a fluorinated copolymer with a tetrafluoroethylene moiety and a vinyl moiety having at least one functional group selected from acetate, alcohol, amine, and amide. The filamentous structures may cooperate to define a plurality of macropores having an average diameter greater than 1 μm and accounting for at least 20% void volume.

In Example 1, the average diameter of the macropores may be 15 μm to 45 μm, or 17 μm to 44 μm.

In Example 1, the macropores may account for 20% to 80% void volume, or 34% to 80% void volume.

In Example 1, the average diameter of the macropores may be uniform over a thickness of at least 0.5 mm.

In Example 1, the fluorinated copolymer may be poly(tetrafluoroethylene-co-vinyl acetate) (TFE-VAc) or poly(tetrafluoroethylene-co-vinyl alcohol) (TFE-VOH).

In Example 1, the fluorinated copolymer may have a tetrafluoroethylene moiety mole content of 15.5% to 23.5% and a vinyl moiety mole content of 76.5% to 84.5%.

In Example 1, each filamentous structure may include a plurality of micropores. The micropores may have an average diameter of 1 μm or less, such as 0.1 μm to 0.6 μm, and account for at least 1% void volume, such as 1% to 20% void volume.

In Example 1, the porous material may also include at least one therapeutic agent dissolved within the filamentous structures, physisorbed or chemisorbed to the filamentous structures, bioconjugated to the filamentous structures, or contained within the macropores.

In Example 1, the porous material may be introduced, deposited, or applied to a biological substrate.

In Example 1, the plurality of macropores may be interconnected.

In Example 1, the porous material may be formed from a formulation consisting essentially of the fluorinated copolymer, a biocompatible solvent system, and a therapeutic agent.

According to another example (“Example 2”), a formulation is disclosed including a biocompatible solvent system, and a fluorinated copolymer dissolved in the biocompatible solvent system at a concentration of 2 wt./vol. % to 20 wt./vol. %, the fluorinated copolymer comprising a tetrafluoroethylene moiety and a vinyl moiety with at least one functional group selected from acetate, alcohol, amine, and amide. The biocompatible solvent system may be configured to diffuse from the fluorinated copolymer upon contact with bodily fluids and leave behind a porous mass.

In Example 2, the porous mass may have a gelation storage modulus of 50 Pa to 500,000 Pa.

In Example 2, the biocompatible solvent system may include water.

In Example 2, the formulation may further include a therapeutic agent.

In Example 2, the formulation may consist essentially of the fluorinated copolymer, the biocompatible solvent system, and the therapeutic agent.

In Example 2, the therapeutic agent may be selected from contrast agents, proteins, peptides, anti-coagulants, vascular cell growth inhibitors, protein kinase and tyrosine kinase inhibitors, analgesics, anti-inflammatory agents, cells, mammalian cells, eukaryotes, prokaryotes, somatic cells, germ cells, erythrocytes, platelets, viruses, prions, DNA, RNA, vectors, cellular fractions, mitochondria, anti-neoplastic/antiproliferative/anti-mitotic agents, and anesthetic agents.

In Example 2, the biocompatible solvent system may include at least one of acetic acid, acetone, anisole, 1-butanol, 2-butanol, butyl acetate, tert-butylmethyl ether, dimethyl sulfoxide (DMSO), ethanol, ethyl acetate, ethyl ether, ethyl formate, formic acid, heptane, isobutyl acetate, isopropyl acetate, methyl acetate, 3-methyl-1-butanol, methylethyl ketone, methylisobutyl ketone, 2-methyl-1-propanol, pentane, 1-pentanol, 1-propanol, 2-propanol, propyl acetate, methyl acetate, triethylamine, propylene glycol, polyethylene glycol, polyethylene oxide.

According to yet another example (“Example 3”), a method is disclosed including injecting a formulation including a biocompatible solvent system, and a fluorinated copolymer dissolved in the biocompatible solvent system at a concentration of 2 wt./vol. % to 20 wt./vol. %, into a treatment site including a biological substrate and bodily fluids, and forming the porous mass by diffusing the biocompatible solvent system into the bodily fluids from the fluorinated copolymer. The porous mass may include a plurality of filamentous structures that cooperate to define a plurality of macropores having an average diameter greater than 1 μm and accounting for at least 20% void volume of the porous mass.

In Example 3, the biological substrate may be selected from a patient's heart, vessel, esophagus, stomach, liver, intestines, vertebrae, sinus, brain sulcus, dermal tissue, bone tissue, muscular tissue, or nervous tissue. The biological substrate may be further selected from an organ structure or a tissue structure, such as a bundle, fiber, ganglion, fascicle, perimysium, endomysium, epimysium, sarcolemma, intercalation, extracellular matrix, and the like.

In Example 3, the method may further include anchoring an implanted medical device into the porous mass.

In Example 3, the treatment site may be beneath a papillary muscle of a patient, within a vessel wall of a patient, or between adjacent organ structures or tissue structures of a patient of a patient.

In Example 3, during the injecting step, the formulation may include a therapeutic agent, and during the forming step, the therapeutic agent may be dissolved within the filamentous structures, physisorbed or chemisorbed to the filamentous structures, bioconjugated to the filamentous structures, or contained within the macropores.

According to yet another example (“Example 4”), a method is disclosed including injecting a formulation into a treatment site including a biological substrate and bodily fluids, the formulation including a biocompatible solvent system and a fluorinated copolymer dissolved in the biocompatible solvent system at a concentration of 2 wt./vol. % to 20 wt./vol. %, the fluorinated copolymer including a tetrafluoroethylene moiety and a vinyl moiety with at least one functional group selected from acetate, alcohol, amine, and amide. The method also includes forming a porous mass by diffusing the biocompatible solvent system from the fluorinated copolymer into the bodily fluids, the porous mass including a plurality of filamentous structures that cooperate to define a plurality of macropores having an average diameter greater than 1 μm and accounting for at least 20% void volume of the porous mass. The method further includes anchoring an implanted medical device into the porous mass.

According to yet another example (“Example 5”), a method is disclosed including injecting a formulation into a treatment site between adjacent organ structures or tissue structures of a patient and including bodily fluids, the formulation including a biocompatible solvent system and a fluorinated copolymer dissolved in the biocompatible solvent system at a concentration of 2 wt./vol. % to 20 wt./vol. %, the fluorinated copolymer including a tetrafluoroethylene moiety and a vinyl moiety with at least one functional group selected from acetate, alcohol, amine, and amide. The method also includes forming a porous mass by diffusing the biocompatible solvent system from the fluorinated copolymer into the bodily fluids, the porous mass including a plurality of filamentous structures that cooperate to define a plurality of macropores having an average diameter greater than 1 μm and accounting for at least 20% void volume of the porous mass, the porous mass separating the adjacent organ structures or tissue structures of the patient.

The foregoing Examples are just that and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is a schematic view of a liquid composition in accordance with an embodiment;

FIG. 2 is a schematic view of the liquid composition being delivered to and hardening at a treatment site to form a hardened porous material in accordance with an embodiment;

FIG. 3 is a graphical view showing the hardening process over time;

FIG. 4 is a schematic view of a first application of the porous hardened material strengthening a patient's heart wall to anchor an implanted device in accordance with an embodiment;

FIG. 5 is a schematic view of a second application of the porous hardened material supporting a patient's papillary muscle in accordance with an embodiment;

FIG. 6 is a schematic view a third application of the porous hardened material reinforcing a patient's vessel wall to receive an implanted device in accordance with an embodiment;

FIG. 7 is a schematic view a fourth application of the porous hardened material separating and/or isolating adjacent fascicles of a patient's skeletal muscle in accordance with an embodiment;

FIGS. 8-11 are scanning electron microscope (SEM) images of hardened material samples in accordance with Example D;

FIGS. 12 and 13 are graphical views of rheological data for the hardened material samples in accordance with Example D; and

FIG. 14 is an SEM image of the hardened material in a skeletal muscle in accordance with Example G;

FIGS. 15 and 16 are SEM images of the hardened material in a skeletal muscle in accordance with Example H.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, adjustments made to optimize performance and/or structural parameters in view of differences in measurements associated with other components, particular implementation scenarios, or imprecise adjustment and/or manipulation of objects by a person or machine, for example. In the event it is determined that individuals having ordinary skill in the relevant arts would not readily ascertain values for such reasonably small differences, the terms “about” and “approximately” can be understood to mean plus or minus 10% of the stated value.

Certain terminology is used herein for convenience only. For example, words such as “top”, “bottom”, “upper,” “lower,” “left,” “right,” “horizontal,” “vertical,” “upward,” and “downward” merely describe the configuration shown in the figures or the orientation of a part in the installed position. Indeed, the referenced components may be oriented in any direction. Similarly, throughout this disclosure, where a process or method is shown or described, the method may be performed in any order or simultaneously, unless it is clear from the context that the method depends on certain actions being performed first.

A coordinate system is presented in the Figures and referenced in the description in which the “Y” axis corresponds to a vertical direction, the “X” axis corresponds to a horizontal or lateral direction, and the “Z” axis corresponds to the interior/exterior direction.

### DESCRIPTION OF VARIOUS EMBODIMENTS

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

Liquid Composition

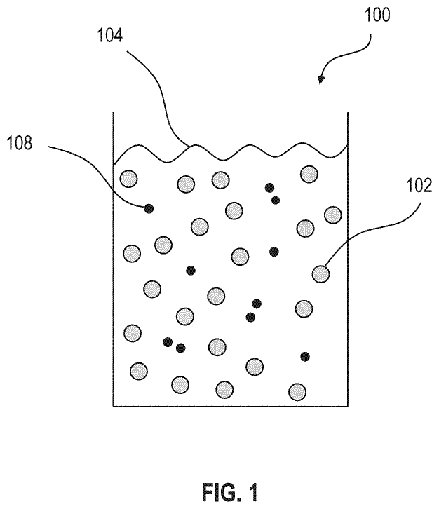

Referring initially to FIG. 1, a liquid composition 100 includes a fluorinated copolymer 102 dissolved or emulsified in a biocompatible solvent system 104. The liquid composition 100 may further include an optional therapeutic agent 108 dissolved or emulsified in the biocompatible solvent system 104. Each element of the liquid composition 100 is described further below.

The fluorinated copolymer 102 of the liquid composition 100 includes a tetrafluoroethylene (TFE) moiety and a vinyl moiety, wherein the vinyl moiety comprises at least one functional group selected from acetate, alcohol, amine, and amide. Suitable fluorinated copolymers 102 include but are not limited to poly(tetrafluoroethylene-co-vinyl acetate) (TFE-VAc), poly(tetrafluoroethylene-co-vinyl alcohol) (TFE-VOH), and/or poly(tetrafluoroethylene-co-vinyl alcohol-co-vinyl[aminobutyraldehydeacetal]) (TFE-VO H-AcAm), for example. The fluorinated copolymer 102 may have a TFE moiety mole content to vinyl moiety mole content of about 10:90, about 20:80, about 30:70, about 40:60, about 50:50, about 60:40, about 70:30, about 80:20, or about 90:10. In certain embodiments, the fluorinated copolymer 102 may have a TFE moiety mole content of about 15.5% to about 23.5%, and a vinyl moiety mole content of about 76.5% to about 84.5%.

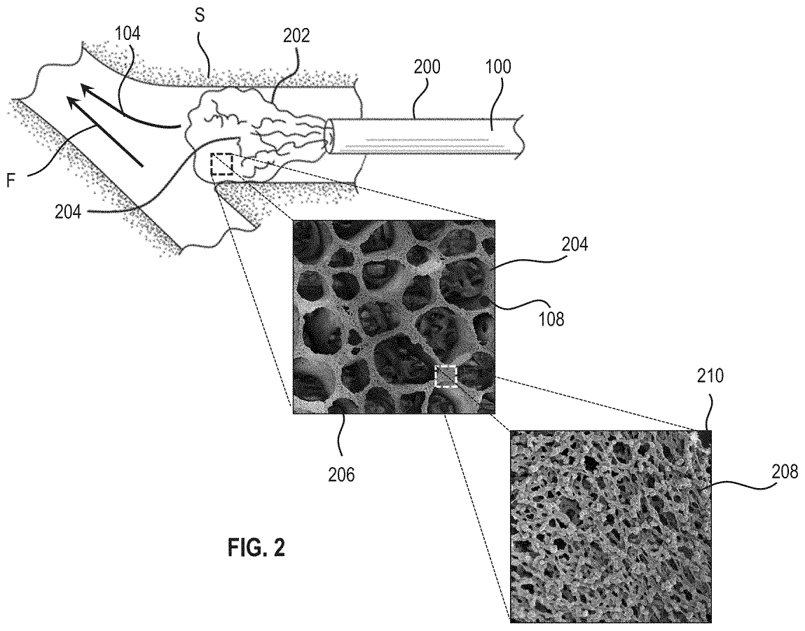

The concentration of the fluorinated copolymer 102 in the biocompatible solvent system 104 (hereinafter, the “solids content”) may vary depending on the intended application. For example, the concentration of the fluorinated copolymer 102 in the biocompatible solvent system 104 may be about 2 wt./vol. % to about 20 wt./vol. %, such as about 2 wt./vol. %, about 4 wt./vol. %, about 6 wt./vol. %, about 8 wt./vol. %, about 10 wt./vol. %, about 12 wt./vol. %, about 14 wt./vol. %, about 16 wt./vol. %, about 18 wt./vol. %, or about 20 wt./vol. %. In certain embodiments, the concentration may be from about 4 wt./vol. % to about 10 wt./vol. %, more specifically from about 4 wt./vol. % to about 8 wt./vol. %. In other embodiments, the concentration may be from about 6 wt./vol. % to about 14 wt./vol. %, more specifically from about 8 wt./vol. % to about 12 wt./vol. %. As discussed further below, the concentration may be controlled to produce a hardened material 202 (FIG. 2) having desired mechanical properties, rheology, porosity, and/or therapeutic effect.

The biocompatible solvent system 104 of the liquid composition 100 may be a low-toxicity, water-miscible solvent that is capable of dissolving the fluorinated copolymer 102. Suitable low-toxicity solvents include “Class 3 Solvents” and/or solvents “Generally Recognized as Safe” (GRAS) as defined by the U.S. Food and Drug Administration (FDA) or the International Conference on Harmonization of Technical Requirements for Registration of Pharmaceuticals for Human Use (ICH). Examples of low-toxicity organic solvents includeacetic acid, acetone, anisole, 1-butanol, 2-butanol, butyl acetate, tert-butylmethyl ether, dimethyl sulfoxide (DMSO), ethanol, ethyl acetate, ethyl ether, ethyl formate, formic acid, heptane, isobutyl acetate, isopropyl acetate, methyl acetate, 3-methyl-1-butanol, methylethyl ketone, methylisobutyl ketone, 2-methyl-1-propanol, pentane, 1-pentanol, 1-propanol, 2-propanol, propyl acetate, methyl acetate, triethylamine, propylene glycol, polyethylene glycol (PG), polyethylene oxide, and the like. In other embodiments, the biocompatible solvent system 104 may include acetonitrile, dioxane, formamide, dimethylformamide, pyridine, N-Methyl-2-pyrrolidone (NMP), methylpyrrolidone, dim ethylacetamide, ethylene glycol, methyoxymethanol, pyridine, piperidine, sulfolane, tetrahydrofuran, trichloroacetic acid, and the like.

Water may be included with the biocompatible solvent system 104 to control the viscosity and/or solvent properties (e.g., dilution) of the liquid composition 100. For example, the biocompatible solvent system 104 may include about 5 vol. %, about 10 vol. %, about 20 vol. %, about 30 vol. %, about 40 vol. %, about 50 vol. %, or more water, as described for example in U.S. Pat. No. 10,092,653.

The optional therapeutic agent 108 may be included in the liquid composition 100 to aid in a therapeutic procedure and/or a therapeutic outcome, whether diagnostic, surgical, or interventional, for example. Suitable therapeutic agents 108 include, for example, contrast agents such as iohexol, iopamidol iopromide, gold nanoparticles, tantalum microparticles, or the like; proteins and peptides such as monoclonal antibodies capable of blocking smooth muscle cell proliferation, inhibitory antibodies, antibodies directed against growth factors, and thymidine kinase inhibitors; anti-coagulants such as D-Phe-Pro-Arg, chloromethyl ketone, an RGD peptide-containing corn pound, heparin, hirudin, antithrombin corn pounds, platelet receptor antagonists, anti-thrombin antibodies, anti-platelet receptor antibodies, prostaglandin inhibitors, platelet inhibitors, antiplatelet peptides, growth factors, such as vascular cell growth promoters such as growth factors, transcriptional activators, translational promotors; vascular cell growth inhibitors such as growth factor inhibitors, growth factor receptor antagonists, transcriptional repressors, translational repressors, replication inhibitors, bi-functional molecules consisting of a growth factor and a cytotoxin, bifunctional molecules consisting of an antibody and a cytotoxin; protein kinase and tyrosine kinase inhibitors (e.g., tyrphostins, genistein, quinoxalines), prostacyclin analogs, cholesterol-lowering agents, statins, angiopoietins, agents that interfere with endogenous vasoactive mechanisms, inhibitors of leukocyte recruitment such as monoclonal antibodies, cytokines, hormones such as β-estradiol 3-(β-D-glucuronide) sodium salt, β-estradiol 3-sulfate sodium salt, -β-estradiol 17-(β-D-glucuronide) sodium salt, estrone 3-sulfate sodium salt, estrone 3-sulfate potassium salt, estradiol acetate, estradiol cypionate; analgesics such as acetylsalicylic acid, α-methyl-4-(isobutyl)phenylacetic acid, diclofenac sodium salt, beta hydroxy acids, salicylic acid, sodium salicylate, naproxen sodium, antibiotics; anti-inflammatory agents such as dexamethasone, dexamethasone sodium phosphate, dexamethasone sodium acetate, estradiol, prednisolone, corticosterone, budesonide, estrogen, sulfasalazine and mesalamine, sirolimus and everolimus (and related analogs), and combination thereof; cells, mammalian cells, eukaryotes, prokaryotes, somatic cells, germ cells, erythrocytes, platelets, viruses, prions, DNA, RNA, vectors, cellular fractions, mitochondria, and the like; anti-neoplastic/antiproliferative/anti-mitotic agents such as paclitaxel, dicumarol, and analogues thereof, rapamycin and analogues thereof, beta-lapachone and analogues thereof, 5-fluorouracil, cisplatin, vinblastine, vincristine, epothilones, endostatin, angiostatin, angiopeptin, and combinations thereof; anesthetic agents such as aspirin, lidocaine, ketamine salt, bupivacaine and ropivacaine, prostaglandin inhibitors, platelet inhibitors, cytotoxic agents such as docetaxel, doxorubicin, paclitaxel, and fluorouracil and analogues thereof, cytostatic agents, cell proliferation effectors, vasodilating agents, cilostazol, carvedilol, antibiotics, sclerosing agent such as ethanol; and combinations thereof.

The liquid composition 100 may include certain additives. One such additive is a contrast medium (e.g., barium salts, iohexol), which may be used for diagnostic or visualization purposes. Another such additive is an energy absorber (e.g., gold nanoparticles), which may be used for targeted thermal ablation.

In accordance with some embodiments, the liquid composition 100 consists only of or consists essentially of the fluorinated copolymer 102, the biocompatible solvent system 104, and the therapeutic agent 108. In other embodiments, the liquid composition 100 consists only of or consists essentially of the fluorinated copolymer 102 and the biocompatible solvent system 104.

The liquid composition 100 may be customized depending on the intended application. For example, the liquid composition 100 may be customized to have a desired viscosity and/or shelf stability while producing the hardened material 202 (FIG. 2) having desired mechanical properties, rheology, porosity, and/or therapeutic effect.

Hardening/Hardened Material

Referring next to FIG. 2, the liquid composition 100 is capable of being injected or otherwise delivered in vivo to a treatment site of a patient. The liquid composition 100 may be delivered from a delivery device 200 (e.g., syringe, catheter) to the treatment site. The treatment site may include the patient's tissue or organ, hereinafter referred to as a biological substrate S. In the illustrated embodiment of FIG. 2, the substrate S is the patient's vessel, but other suitable biological substrates S and applications are described further below.

After being delivered to substrate S as shown in FIG. 2, the liquid composition 100 may contact blood or other bodily fluids F. In a process referred to herein as “hardening”, “gelation”, or “curing”, the biocompatible solvent system 104 (FIG. 1) of the liquid composition 100 dissipates into the patient's body, the substrate S, and/or the bodily fluids F, and the water-insoluble fluorinated copolymer 102 (FIG. 1) of the liquid composition 100 precipitates and/or gels at the treatment site to form the hardened material 202. The term “hardened material” is meant to define a fluorinated copolymer that is precipitated and/or gelled to a coherent mass or a coherent porous mass in a solid state or a gel state.

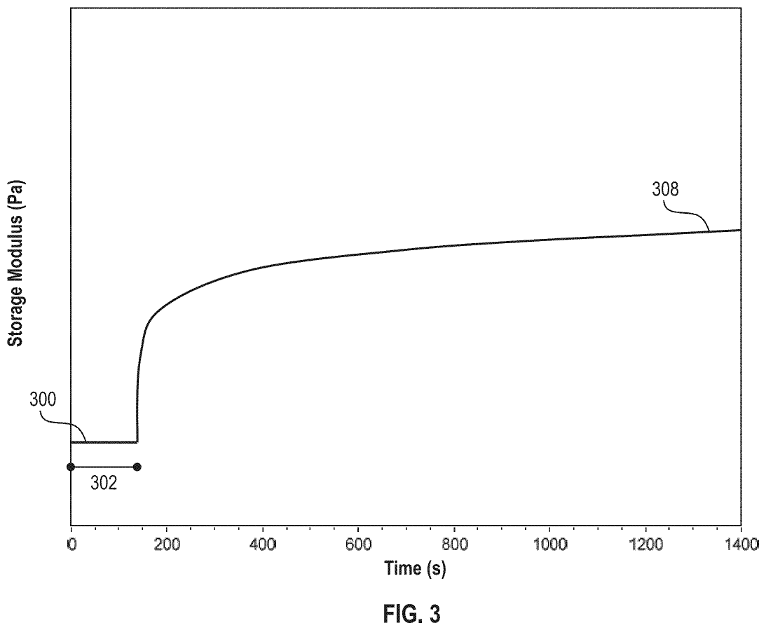

Referring next to FIG. 3, the hardening process is represented graphically over time. During delivery, the liquid composition 100 has an initial rheological gelation storage modulus 300. After delivery, there may be an activation period 302 before hardening begins. The duration of the activation period 302 may vary. In some embodiments, the activation period 302 may be about 60 seconds, about 90 seconds, about 120 seconds, about 150 seconds, about 180 seconds, about 210 seconds, about 240 seconds, or longer, for example. After the activation period 302, the material may progress continuously from the initial rheological gelation storage modulus 300 of the liquid composition 100 to a final rheological gelation storage modulus 308 of the hardened material 202 (as shown in FIG. 3).

Returning to FIG. 2, the resulting hardened material 202 may be a viscoelastic material and may be present in a coherent mass or a coherent porous mass in a solid state or a gel state. The final rheological gelation storage modulus of the hardened material 202 may vary depending on the intended application and the intended substrate S (FIG. 2). For example, the gelation storage modulus of the hardened material 202 may be about 50 Pa to about 500,000 Pa (500 kPa), such as about 50 Pa, about 100 Pa, about 500 Pa, about 1,000 Pa, about 5,000 Pa, about 10,000 Pa, about 50,000 Pa, about 100,000 Pa, about 200,000 Pa, or about 500,000 Pa. In some embodiments, the gelation storage modulus of the hardened material 202 is about 10,000 Pa to about 200,000 Pa. The final rheological gelation storage modulus of the hardened material 202 may match the softness or hardness of the intended substrate S (FIG. 2). For example, a low modulus of about 50 Pa to about 500 Pa may be desired when the intended substrate S is very soft (e.g., brain), a moderate modulus of about 500 Pa to about 5,000 Pa may be desired when the intended substrate S is soft (e.g., heart, blood vessels), and a high modulus of about 5,000 Pa to about 500,000 Pa may be desired when the intended substrate S is hard (e.g., vertebrae).

The hardened material 202 may be a porous mass having a macroporous and/or microporous structure. The pore structure and the porosity of the hardened material 202 may vary depending on the intended application. In certain embodiments, the hardened material 202 may be a coherent isotropic porous mass, such as a bolus or a plug, with a porosity substantially uniform over the hardened material 202. In other embodiments, the hardened material 202 may be a diffuse anisotropic porous mass, such as discrete particles with diameters of about 100 μm or less or thin matrices with thicknesses of about 100 μm or less, with a porosity substantially uniform across the diffuse mass. The porous nature of the hardened material 202 may promote biocompatibility and tissue ingrowth in certain embodiments.

The macroporous structure may include a plurality of fluorinated copolymer filamentous structures 204 that cooperate to define a plurality of interconnected or disconnected macropores 206, as shown in FIG. 2. The average diameter of the macropores 206 may be greater than about 1 μm, such as about 5 μm, about 10 μm, about 20 μm, about 30 μm, about 40 μm, about 50 μm, or larger. In some embodiments, the average diameter of the macropores 206 is about 10 μm to about 50 μm, more specifically about 15 μm to about 45 μm, more specifically about 20 μm to about 25 μm. The macropores 206 may account for about 20% or more of the hardened material 202 (which may be measured as a pore area ratio), such as about 20%, about 30%, about 40%, about 50%, about 60%, about 70%, about 80%, about 90%, or more of the hardened material 202. In certain embodiments, the macropores 206 account for about 20% to about 80% of the hardened material 202, more specifically about 30% to about 50%.

The microporous structure may include a plurality of fluorinated copolymer filamentous structures 208 that cooperate to define a plurality of interconnected or disconnected micropores 210, as shown in FIG. 2. The average diameter of the micropores 210 may be about 1 μm or less, such as about 0.01 μm, about 0.1 μm, about 0.2 μm, about 0.4 μm, about 0.6 μm, about 0.8 μm, or about 1 μm. In some embodiments, the average diameter of the micropores 210 is about 0.1 μm to about 0.6 μm, more specifically about 0.1 μm to about 0.2 μm. The micropores 210 may account for about 1% or more of the hardened material 202 (which may be measured as a pore area ratio), such as about 1%, about 5%, about 10%, about 15%, about 20%, about 25% or more of the hardened material 202. In certain embodiments, the micropores 210 account for about 1% to about 20% of the hardened material 202.

In certain embodiments, the hardened material 202 may have a combined macroporous/microporous structure, with the micropores 210 being present in the fluorinated copolymer filamentous structures 204 that surround the macropores 206. In this arrangement, each fluorinated copolymer filamentous structure 204 may comprise a plurality of smaller fluorinated copolymer filamentous structures 208 and their corresponding micropores 210. Thus, the larger macropores 206 may be surrounded by a plurality of smaller micropores 210.

As noted above, the liquid composition 100 may be customized to control the mechanical properties, rheology, porosity, and/or therapeutic effect of the hardened material 202. For example, a first liquid composition 100 having a lower concentration of fluorinated copolymer 102 (e.g., 4 wt./vol. %) may produce a hardened material 202 having more void volume and larger pores than a second liquid composition 100 having a higher concentration of fluorinated copolymer 102 (e.g., 6 wt./vol. %). In this way, the porosity of the hardened material 202 may be controlled by varying the concentration and other properties of the liquid composition 100.

If a therapeutic agent 108 is present in the liquid composition 100, the therapeutic agent 108 may also be present in the hardened material 202. At least initially, the therapeutic agent 108 may be dissolved within the fluorinated copolymer filamentous structures 204, 208, physisorbed or chemisorbed to the fluorinated copolymer filamentous structures 204, 208, bioconjugated to the fluorinated copolymer filamentous structures 204, 208 and/or contained within the macropores 206 and/or micropores 210 of the hardened material 202. Over time, some or all of the therapeutic agent 108 may disperse from the hardened material 202 and into the patient. In another embodiment, over time, some or all of the therapeutic agent 108 may not disperse from the hardened material 202 and into the patient.

Medical Applications

The hardened material 202 may be delivered to different treatment sites for different medical applications. Suitable biological substrates S configured to receive the hardened material 202 are found throughout the patient's body, including the patient's cardiovascular system (e.g., the pericardium, pericardial space, myocardium, or papillary muscle of the heart), vascular system (e.g., the intima, media, or adventitia of a vessel), muscular system (e.g., skeletal muscle tissue, cardiac muscle tissue, smooth muscle tissue), and nervous system (e.g., cardiac nerves, peripheral nerves). Other suitable biological substrates S include, for example, the patient's esophagus, stomach, liver, intestines, vertebrae, sinus, brain sulcus, dermal tissue, and any other biological tissues or organs. Other suitable biological substrates S include, for example, organ structures and tissue structures, for example, a bundle, ganglion, fiber, fascicle, perimysium, endomysium, epimysium, sarcolemma, intercalation, extracellular matrix, and any other structures. Depending on the desired application, the hardened material 202 may strengthen, support, move, reinforce, separate, isolate, and/or bulk the substrate S.

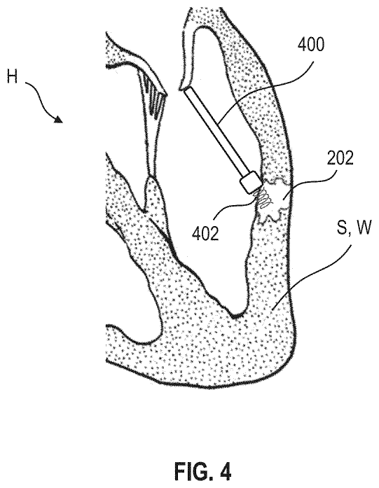

In the illustrated embodiment of FIG. 4, the substrate S is a wall W of a patient's heart H. An implanted device is provided in the form of an artificial cord 400 having an anchor end 402 (e.g., a helical screw). The hardened material 202 may be introduced, deposited, or otherwise applied at one or more locations within the patient's heart wall W using methods known in the art, such as within the pericardium, the pericardial space, and/or the myocardium of the patient's heart wall W. Anchoring the anchor end 402 into the hardened material 202 may strengthen the connection between the artificial cord 400 and the heart wall W and resist pull-out forces acting on the artificial cord 400. In other embodiments, the hardened material 202 may be used to strengthen other substrates S and/or to anchor other implanted devices.

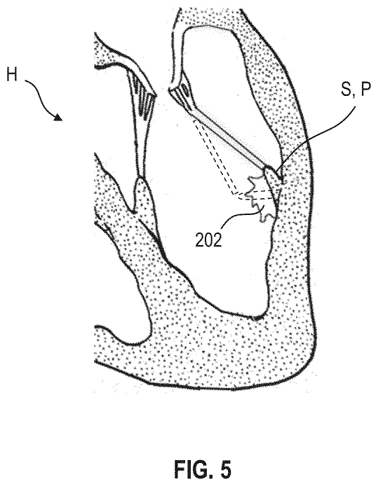

In the illustrated embodiment of FIG. 5, the substrate S is the papillary muscle P of a patient's enlarged heart H. The papillary muscle P is susceptible to drooping downward to a horizontal orientation (shown in phantom lines in FIG. 5), which is also referred to as “tethering.” The hardened material 202 may be positioned beneath the papillary muscle P to move and support the papillary muscle P in its normal, vertical orientation (shown in solid lines in FIG. 5), thereby serving as a buttress for the papillary muscle P. The hardened material 202 may be located internally and/or externally relative to the papillary muscle P. In other embodiments, the hardened material 202 may be used to move and/or support other substrates S.

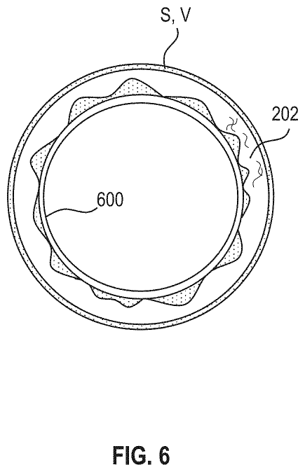

In the illustrated embodiment of FIG. 6, the substrate S is a patient's weakened vessel wall V having low radial and/or circumferential strength. The hardened material 202 may be introduced, deposited, or otherwise applied at one or more locations within the vessel wall V using methods known in the art, such as within the intima, media, and/or adventitia of the vessel wall V, thereby reinforcing the vessel wall V. The hardened material 202 may inhibit progression of vascular disease through the vessel wall V. In some applications, the hardened material 202 may also support an implanted device against the vessel wall V, such as the illustrated stent-graft 600 of FIG. 6. In other embodiments, the hardened material 202 may be used to reinforce other substrates S.

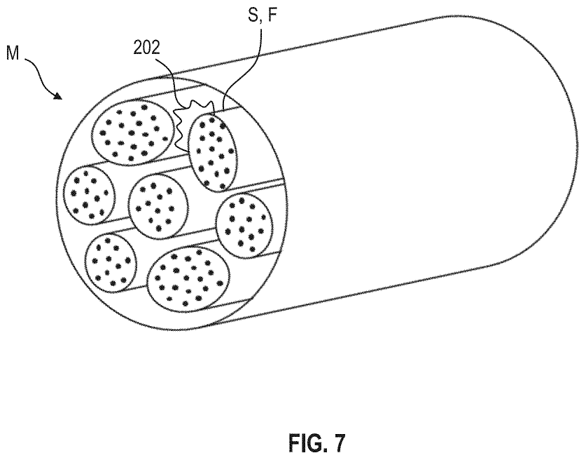

In the illustrated embodiment of FIG. 7, the substrate S is a fascicle F of a patient's skeletal muscle M. The hardened material 202 may be positioned between one or more fascicles F to separate and/or isolate adjacent fascicles F. In this way, the hardened material 202 may promote movement (e.g., contraction, sliding) between adjacent fascicles F with reduced stretching of the fascicles F. The hardened material 202 may also break apart scar tissue or other obstructions between adjacent fascicles F. Thus, the hardened material 202 may induce healing and sarcomerogenesis via extracellular matrix remodeling, including for infarct. Although the patient's skeletal muscle M is shown in FIG. 7, such separation and/or isolation may also be performed in cardiac muscle tissue or smooth muscle tissue. Additionally, such separation and/or isolation may also be performed in nervous tissue, such as in cardiac nerves to mitigate fascicle collapse to restore normal cell-to-cell conduction and signal transmission, and in peripheral nerves to target and interface for neural-enabled prostheses and to reduce neuralgia.

Other applications of the hardened material 202 include, for example, bulking a valve annulus and filling a vessel dissection, such as a false aortic lumen dissection.

The applications shown in FIGS. 4-7 are provided as examples of the various features of the present disclosure and, although the combination of those illustrated features is clearly within the scope of invention, those examples and its illustrations are not meant to suggest the inventive concepts provided herein are limited from fewer features, additional features, or alternative features to one or more of those features shown in FIGS. 4-7. For example, in various embodiments, the anchoring features described with reference to FIG. 4 may also include the reinforcement features described with reference to FIG. 6. It should also be understood that the reverse is true as well. For another example, in various embodiments, the hardened material 202 may comprise a therapeutic agent 108, wherein over time, some or all of the therapeutic agent 108 may disperse from the hardened material 202 and into the patient. For another example, in various embodiments, the hardened material 202 may comprise a therapeutic agent 108, wherein over time, some or all of the therapeutic agent 108 does not disperse from the hardened material 202 and into the patient.

### EXAMPLES

### Example A: Syntheses of Fluorinated Copolymers Comprising Tetrafluoroethylene and Functional Groups Comprising Vinyl Acetate (TFE-VAc)

Copolymers comprising varying mole ratios of vinyl acetate to tetrafluoroethylene (VAc:TFE) were prepared according the following general synthetic scheme. To a nitrogen purged 1 L pressure reactor under vacuum were added 500 g DI water, 2.0 g of 20% aqueous surfactant, 30 ml of distilled vinyl acetate, 10 g of n-butanol, and 0.2 g of ammonium persulfate. Tetrafluoroethylene monomer was then fed into the reactor until the reactor pressure reached 1500 KPa. The mixture was stirred and heated to 50° C. When a pressure drop was observed, 25 ml of additional vinyl acetate was slowly fed into the reactor. The reaction was stopped when the pressure dropped another 150 KPa after vinyl acetate addition. The copolymer was obtained from freeze-thaw coagulation of the latex emulsion, cleaned with methanol/water extraction, and air dried.

The copolymers' composition and molecular weight are listed in Table 1 below.

TABLE 1

| Copolymer# | VAc mole % | TFE mole % | MW (KDa) |
|---|---|---|---|
| 100-0 | 80.0 | 20.0 | 300 |
| 100-1 | 81.1 | 18.9 | 337 |
| 100-2 | 81.2 | 18.8 | 220 |
| 100-3 | 84.5 | 15.5 | 430 |
| 100-4 | 76.5 | 23.5 | 122 |

### Example B: Synthesis of a Fluorinated Copolymer Comprising Tetrafluoroethylene and Functional Groups Comprising Alcohol (TFE-VOH)

The vinyl acetate groups of copolymer #100-0 of Example A were hydrolyzed to vinyl alcohol as follows. To a 50 ml round bottle flask were added 0.5 g of copolymer #100-0 (predissolved in 10 ml methanol) and 0.46 g NaOH (predissolved in 2 ml DI water). The mixture was stirred and heated to 60° C. for 5 hrs. The reaction mixture was then acidified to pH 4, precipitated in DI water, dissolved in methanol, again precipitated in DI water, and air dried. The resulting product was a copolymer of TFE-VOH.

### Example C: Preparation of Fluorinated Copolymer Liquid Compositions

Eight different liquid compositions were prepared according to Table 2 below using the fluorinated copolymer #100-0 (TFE-VAc) of Example A, and the fluorinated copolymer (TFE-VOH) of Example B. Briefly, the fluorinated copolymer was added to the biocompatible solvent system in a vial, the vial headspace purged with nitrogen then capped, the capped vial placed into a 60° C. oven, the capped vial gently tumbled for 24 hours and cooled to room temperature, to produce the fluorinated copolymer liquid composition.

TABLE 2

| | Fluorinated | | |
|---|---|---|---|
| | Copolymer | Solids Content | Biocompatible |
| Sample ID | Type | (wt./vol. %) | Solvent System |
| P1 | TFE-VAc | 4 | DMSO |
| P2 | | 6 | |
| PP8 | | 8 | |
| P3 | | 10 | |
| P4 | | 14 | |
| P22 | TFE-VOH | 10 | 60 vol. % |
| | | | DMSO:40 |
| | | | vol. % water |
| P17 | | 4 | PG |
| P19 | | 10 | |

### Example D: Impact of Fluorinated Copolymer Liquid Composition on Porosity and Gelation Storage Modulus

Rheology of the fluorinated copolymer liquid compositions of Example C during the hardening process into coherent masses, was measured as follows. The liquid composition was placed onto a 25 mm plate of a rheometer (TA DHR-2, TA Instruments, New Castle, Del.), the cone was applied in oscillation mode at a frequency of 10 rad/sec at a stress of 5 uN-m, and the storage modulus of the sample was measured over a time sweep of 1200 sec, as water was injected into the cone-plate gap to initiate hardening of the coherent mass.

Porosity of the fluorinated copolymer liquid compositions of Example C in the form of hardened coherent thin films, was measured as follows. Approximately 5 ml of the liquid composition was poured onto a clean glass plate, a casting knife (BYK, Columbia, Md.) was drawn across the liquid composition, the glass plate was immersed into deionized water or saline for at least 4 hrs and removed, the hardened film was gently lifted from the glass plate, and the hardened film was air dried at room temperature, to produce a dry hardened film of about 25 um to about 100 um thick.

The dry hardened films were imaged under scanning electron microscopy (Hitachi SU8200) and analyzed using Image J image analysis software (National Institutes of Health, USA), using methods well known to the art for measuring void volume and pore size. Briefly, the SEM images were scanned for pores over a total area of about 25 μm²; the ratio of the pore areas to the about 25 μm² total area constituted a micro-void volume, and the average diameters of the pores constituted the micro-pore diameter. Additionally, the SEM images were scanned for pores over a total area of about 0.1 mm²; the ratio of the pore areas to the about 0.1 mm² total area constituted a macro-void volume, and the average diameters of the pores constituted a macro-pore diameter.

Table 3 provides the calculated void volumes, pore sizes, and storage moduli. As can be seen from the data, the void volume and pore size for the TFE-VAc samples were negatively correlated to the solids content, while the storage modulus was positively correlated to the solids content. The void volume and pore size for the TFE-VOH samples were approximately independent of the biocompatible solvent system, while the storage moduli remained of the same approximate magnitude.

TABLE 3

| | | Fluorinated Copolymer Type | | | | | | | |
|---|---|---|---|---|---|---|---|---|---|
| | | TFE-VAc | | | | | TFE-VOH | | |
| | | Biocompatible Solvent System | | | | | | | |
| | | | | | | | DMSO:Water | | |
| | | DMSO | | | | | (60:40, v:v) | PG | |
| | | Solids Content (wt./vol. %) | | | | | | | |
| | | 4 | 6 | 8 | 10 | 14 | 10 | 4 | 10 |
| | | Sample ID | | | | | | | |
| | | P1 | P2 | PP8 | P3 | P4 | P22 | P17 | P19 |
| Average | Macro | 25 | 17 | none | none | None | 44 | none | 33 |
| Pore Size | Micro | 0.14 | ~0.14 | 0.55 | ~0.55 | 0.55 | none | none | none |
| Diameter | | | | | | | | | |
| (μm) | | | | | | | | | |
| Pore Area | Macro | 50 | 34 | N/A | N/A | N/A | 60 | N/A | 80 |
| Ratio (%) | Micro | 20 | ~20 | 20 | ~1 | 1 | N/A | N/A | N/A |

| | | | | | | | | |
|---|---|---|---|---|---|---|---|---|
| Rheology Gelation | 30 | 170 | 300 | 340 | 490 | 2.5 | 0.08 | 0.7 |
| Storage Modulus | | | | | | | | |
| (kPa) | | | | | | | | |

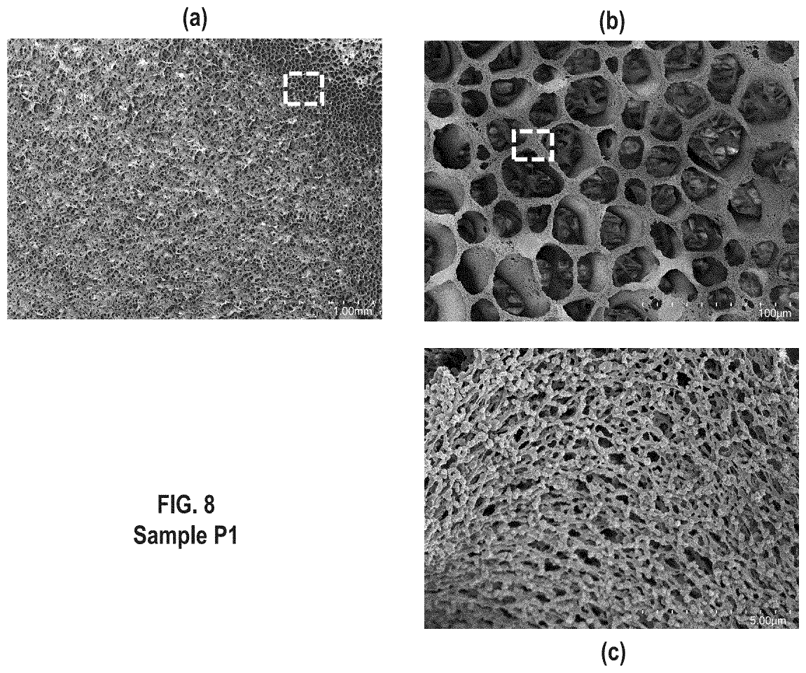

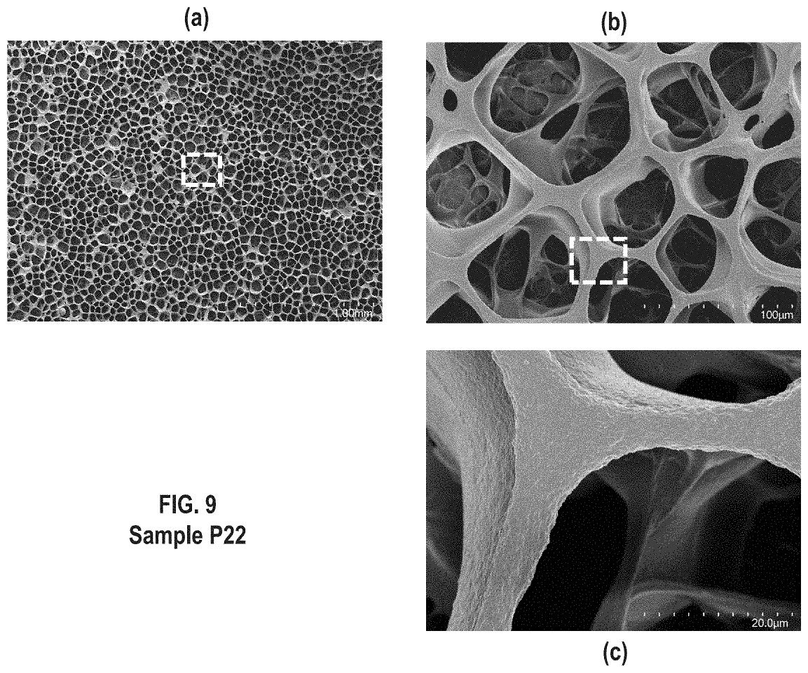

Four of the samples—specifically Sample P1 (FIGS. 8(a) and (b)), Sample P2, Sample P22 (FIGS. 9(a) and (b)), and Sample P19— had macroporous structures. Of these four samples, the two TFE-VAc samples—specifically Sample P1 (FIG. 8(c)) and Sample P2— also had microporous structures with micropores present in the fluorinated copolymer filamentous structures surrounding the macropores, whereas the two TFE-VOH samples—specifically Sample P22 (FIG. 9(c)) and Sample P19— only had macroporous structures with no additional porosity visible in the fluorinated copolymer filamentous structures surrounding the macropores.

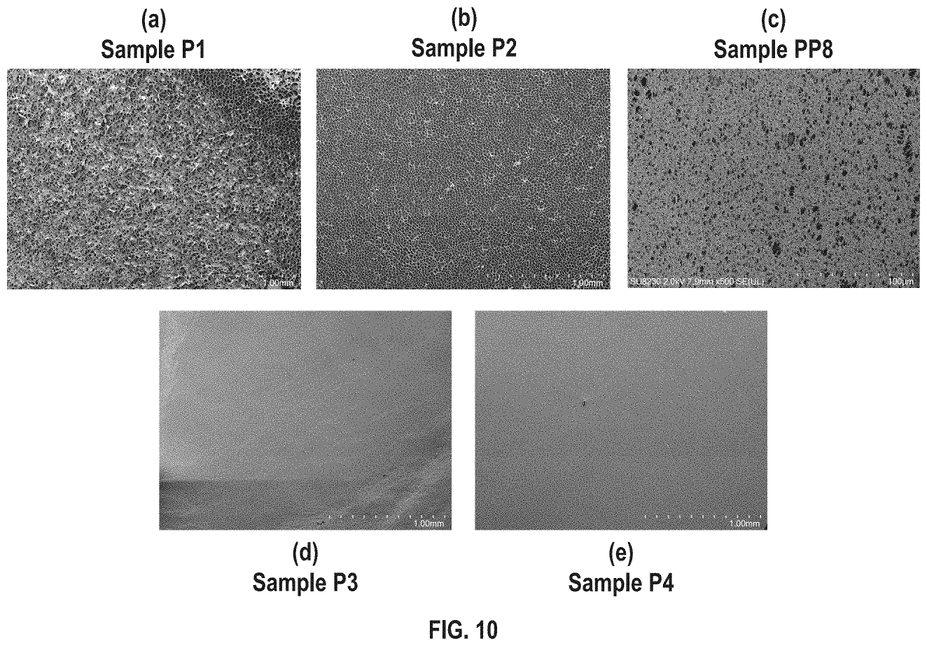

As shown in Table 3 and in FIGS. 10(a)-(e), the percent solids content had an indirect impact on porosity for the TFE-VAc samples. Sample P1 had the lowest percent solids content and the highest macro-porosity (FIG. 10(a)). Sample P2 had a higher percent solids content than Sample P1 and less macro-porosity (FIG. 10(b)). Sample PP8 had a higher percent solids content than sample P2 and no measurable macro-porosity, only micro-porosity (FIG. 10(c)). Sample P4 had the highest percent solids and the lowest macro- and micro-porosities (FIG. 10(e)).

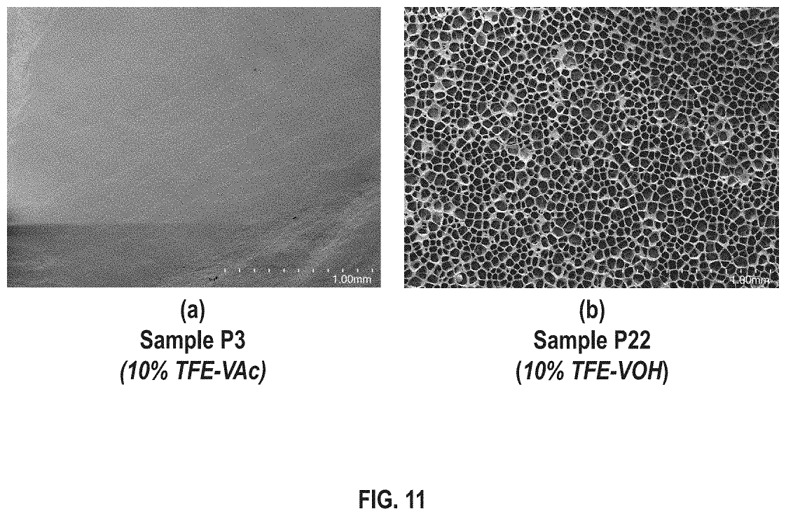

As shown in Table 3 and in FIGS. 11(a)-(b), the fluorinated copolymer type also impacted porosity. Sample P3 comprised 10% TFE-VAc solids and had about 0% macro-porosity and 0.5% micro-porosity (FIG. 11(a)), whereas Sample P22 comprised 10% TFE-VOH solids and had 44% macro-porosity (FIG. 11(b)) and no measurable micro-porosity (FIG. 9(c)).

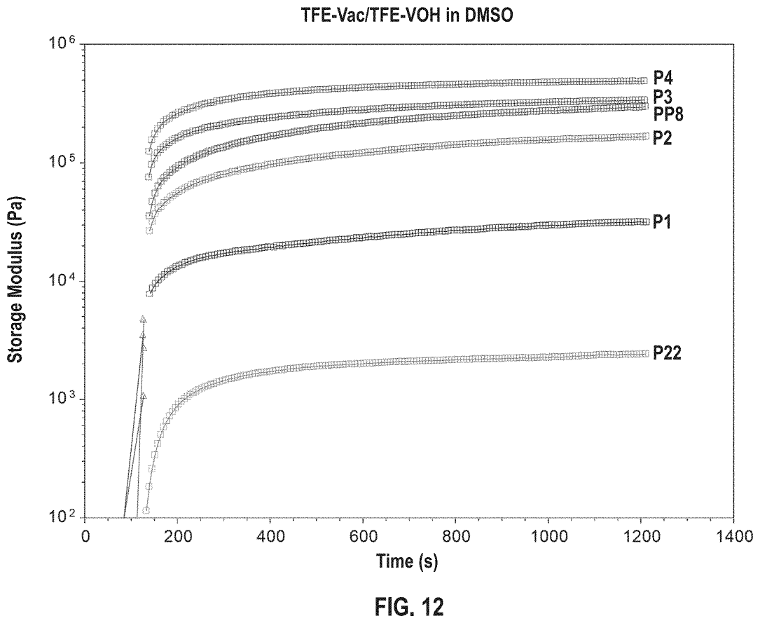

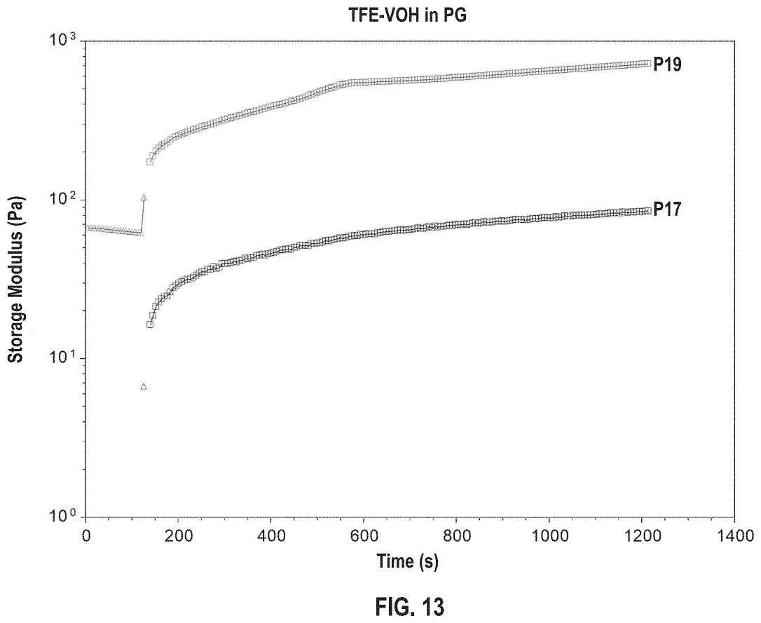

As shown in Table 3 and FIGS. 12 and 13, the percent solids content had a direct impact on storage modulus. Sample P4 had a high percent solids content and a high modulus, whereas sample P1 had a low percent solids content and a low modulus. The fluorinated copolymer type also impacted storage modulus. The storage modulus of Sample P3 comprising 10% TFE-VAc solids had a storage modulus about two orders of magnitude higher than Sample P22 comprising 10% TFE-VOH.

### Example E: Preparation of an Injectable Formulation of TFE-VOH

The TFE-VOH of Example B was dissolved in propylene glycol at 80° C. with gentle tumbling for about 48-72 hours. Phosphate buffered saline (Invitrogen) was added at a concentration of 60:40 v/v at 70° C. with gentle tumbling for about 24 hours to form an injectable formulation comprising 8% w/v TFE-VOH.

### Example F: Preparation of a Sterile Pre-Filled Syringe Comprising a Solution of TFE-VOH

The injectable formulation of Example E was drawn into a 3 ml sterile disposable luer-lok syringe (Beckton-Dickinson) to the 1.5 ml mark. The syringe was then sealed with a luer-threaded cap (ThermoFisher). The capped syringe was steam sterilized. After sterilization and cooling, a sterile needle was attached to the pre-filled syringe. The result was a sterile pre-filled syringe comprising a solution of TFE-VOH.

### Example G: In Vivo Injection of a Porous Hardened Material Comprising TFE-VOH into a Skeletal Muscle

The TFE-VOH solution from the sterile pre-filled syringe of Example F was injected into a spinalis muscle, with the needle insertion oriented approximately parallel to the muscle fibers. The formulation was allowed to harden in place for 2 hours, after which the spinalis muscle was evaluated for H&E histology.

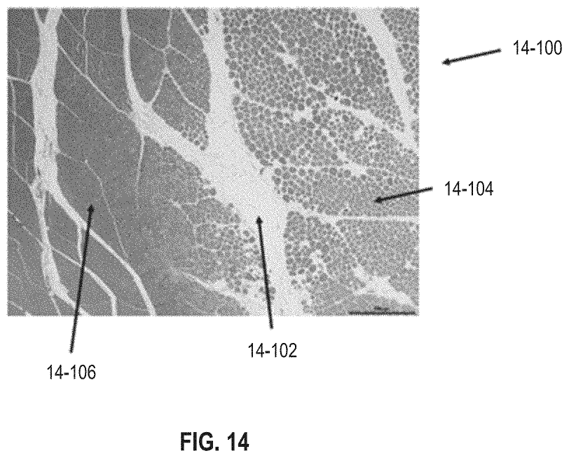

FIG. 14 shows the architecture of the muscle 14-100 at the boundary of the injection site 14-102. Muscle 14-106 that was not injected showed normal tissue architecture. Muscle 14-104 that was injected with the TFE-VOH was observed to have the TFE-VOH polymer present around the endomysium and separating the individual myocytes.

### Example H: In Vivo Implantation of a Porous Hardened Material Comprising TFE-VOH into a Skeletal Muscle

To verify the thickness of a target muscle and orientation of its muscle fibers, the muscle was pre-scanned using ultrasound (Vivid IQ; 35 frames/sec, frequency 4.0/8/0 MHz, 2.5 cm depth).

Using ultrasound guidance, the needle from the sterile pre-filled syringe of Example F was oriented parallel to the muscle fibers. The contents of the syringe were injected over a 5 second duration. The formulation was allowed to harden for 1 hour. The muscle was evaluated for H&E histology and for cryosection histology.

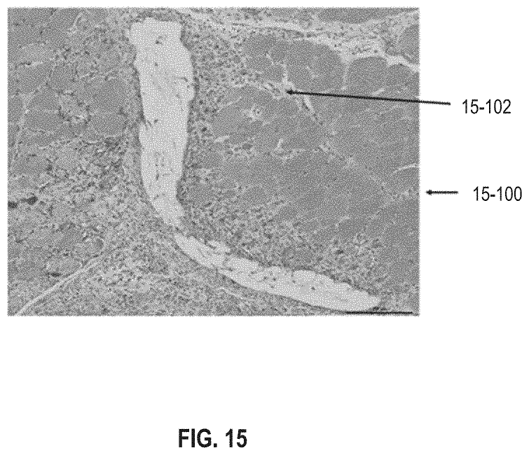

FIG. 15 shows the healing response of the muscle 15-100 to the injected TFE-VOH, as evaluated by H&E histology. A minimal-to-mild inflammatory response with myocyte degeneration/regeneration was observed, with expanded perimysium and epimysium spaces 15-102. In contrast, a control injection with saline did not show expansion of the perimysium or epimysium spaces.

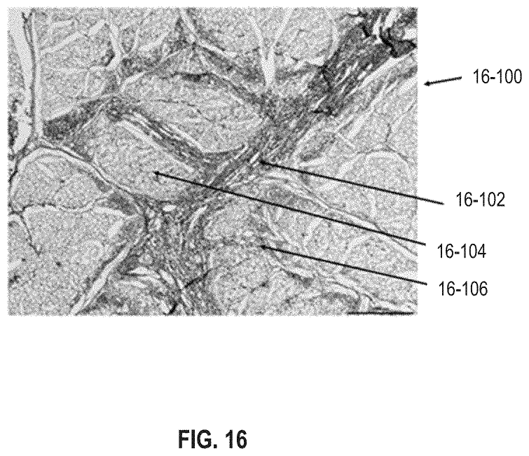

FIG. 16 shows the structure of the TFE-VOH in the muscle 16-100 as evaluated by cryosection histology. TFE-VOH 16-102 was observed to have flowed and separated individual muscle fascicles 16-104 and was often identified in the expanded perimysium and epimysium spaces 16-106. In contrast, a control injection with saline did not show separation of muscle fascicles nor expansion of the perimysium or epimysium spaces.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. A porous material comprising:
a plurality of filamentous structures comprising a fluorinated copolymer with a tetrafluoroethylene moiety and a vinyl moiety having at least one functional group selected from acetate, alcohol, amine, and amide, the filamentous structures cooperating to define a plurality of macropores having an average diameter greater than 1 μm and accounting for at least 20% void volume.

2. The porous material of claim 1, wherein the average diameter of the macropores is 15 μm to 45 μm.

3. The porous material of claim 2, wherein the average diameter of the macropores is 17 μm to 44 μm.

4. The porous material of claim 1, wherein the macropores account for 20% to 80% void volume.

5. The porous material of claim 4, wherein the macropores account for 34% to 80% void volume.

6. The porous material of claim 1, wherein the average diameter of the macropores is uniform over a thickness of at least 0.5 mm.

7. The porous material of claim 1, wherein the fluorinated copolymer is one of poly(tetrafluoroethylene-co-vinyl acetate) (TFE-VAc) and poly(tetrafluoroethylene-co-vinyl alcohol) (TFE-VOH).

8. The porous material of claim 1, wherein the fluorinated copolymer has a tetrafluoroethylene moiety mole content of 15.5% to 23.5% and a vinyl moiety mole content of 76.5% to 84.5%.

9. The porous material of claim 1, wherein each filamentous structure comprises a plurality of micropores.

10. The porous material of claim 9, wherein the micropores have an average diameter of 1 μm or less and account for at least 1% void volume.

11. The porous material of claim 9, wherein the micropores have an average diameter of 0.1 μm to 0.6 μm and account for 1% to 20% void volume.

12. The porous material of claim 1, further comprising at least one therapeutic agent dissolved within the filamentous structures, physisorbed or chemisorbed to the filamentous structures, bioconjugated to the filamentous structures, or contained within the macropores.

13. The porous material of claim 1, wherein the porous material is introduced, deposited, or applied to a biological substrate.

14. The porous material of claim 1, wherein the plurality of macropores are interconnected.

15. The porous material of claim 1, wherein the porous material is formed from a formulation consisting essentially of the fluorinated copolymer, a biocompatible solvent system, and a therapeutic agent.

16. A formulation comprising:
a biocompatible solvent system; and
a fluorinated copolymer dissolved in the biocompatible solvent system at a concentration of 2 wt./vol. % to 20 wt./vol. %, the fluorinated copolymer comprising a tetrafluoroethylene moiety and a vinyl moiety with at least one functional group selected from acetate, alcohol, amine, and amide,
wherein the biocompatible solvent system is configured to diffuse from the fluorinated copolymer upon contact with bodily fluids and leave behind a porous mass.

17. The formulation of claim 16, wherein the porous mass has a gelation storage modulus of 50 Pa to 500,000 Pa.

18. The formulation of claim 16, wherein the biocompatible solvent system includes water.

19. The formulation of claim 16, further comprising a therapeutic agent.

20. The formulation of claim 19, wherein the formulation consists essentially of the fluorinated copolymer, the biocompatible solvent system, and the therapeutic agent.

21. The formulation of claim 19, wherein the therapeutic agent is selected from contrast agents, proteins, peptides, anti-coagulants, vascular cell growth inhibitors, protein kinase and tyrosine kinase inhibitors, analgesics, anti-inflammatory agents, cells, mammalian cells, eukaryotes, prokaryotes, somatic cells, germ cells, erythrocytes, platelets, viruses, prions, DNA, RNA, vectors, cellular fractions, mitochondria, anti-neoplastic/antiproliferative/anti-mitotic agents, and anesthetic agents.

22. The formulation of claim 16, wherein the biocompatible solvent system includes at least one of acetic acid, acetone, anisole, 1-butanol, 2-butanol, butyl acetate, tert-butylmethyl ether, dimethyl sulfoxide (DMSO), ethanol, ethyl acetate, ethyl ether, ethyl formate, formic acid, heptane, isobutyl acetate, isopropyl acetate, methyl acetate, 3-methyl-1-butanol, methylethyl ketone, methylisobutyl ketone, 2-methyl-1-propanol, pentane, 1-pentanol, 1-propanol, 2-propanol, propyl acetate, methyl acetate, triethylamine, propylene glycol, polyethylene glycol, polyethylene oxide.

23. A method comprising:
injecting the formulation of claim 16 into a treatment site including a biological substrate and bodily fluids; and
forming the porous mass by diffusing the biocompatible solvent system into the bodily fluids from the fluorinated copolymer, the porous mass comprising a plurality of filamentous structures that cooperate to define a plurality of macropores having an average diameter greater than 1 μm and accounting for at least 20% void volume of the porous mass.

24. The method of claim 23, wherein the biological substrate is selected from a patient's heart, vessel, esophagus, stomach, liver, intestines, vertebrae, sinus, brain sulcus, dermal tissue, bone tissue, muscular tissue, nervous tissue, bundle, fiber, ganglion, fascicle, perimysium, endomysium, epimysium, sarcolemma, intercalation, or extracellular matrix.

25. The method of claim 23, further comprising anchoring an implanted medical device into the porous mass.

26. The method of claim 23, wherein the treatment site is beneath a papillary muscle of a patient.

27. The method of claim 23, wherein the treatment site is within a vessel wall of a patient.

28. The method of claim 23, wherein the treatment site is between adjacent organ structures or tissue structures of a patient.

29. The method of claim 23, wherein:
during the injecting step, the formulation comprises a therapeutic agent; and
during the forming step, the therapeutic agent is dissolved within the filamentous structures, physisorbed or chemisorbed to the filamentous structures, bioconjugated to the filamentous structures, or contained within the macropores.

30. A method comprising:
injecting a formulation into a treatment site including a biological substrate and bodily fluids, the formulation comprising:
a biocompatible solvent system; and
a fluorinated copolymer dissolved in the biocompatible solvent system at a concentration of 2 wt./vol. % to 20 wt./vol. %, the fluorinated copolymer comprising a tetrafluoroethylene moiety and a vinyl moiety with at least one functional group selected from acetate, alcohol, amine, and amide;
forming a porous mass by diffusing the biocompatible solvent system from the fluorinated copolymer into the bodily fluids, the porous mass comprising a plurality of filamentous structures that cooperate to define a plurality of macropores having an average diameter greater than 1 μm and accounting for at least 20% void volume of the porous mass; and
anchoring an implanted medical device into the porous mass.

31. A method comprising:
injecting a formulation into a treatment site between adjacent organ structures or tissue structures of a patient and including bodily fluids, the formulation comprising:
a biocompatible solvent system; and
a fluorinated copolymer dissolved in the biocompatible solvent system at a concentration of 2 wt./vol. % to 20 wt./vol. %, the fluorinated copolymer comprising a tetrafluoroethylene moiety and a vinyl moiety with at least one functional group selected from acetate, alcohol, amine, and amide; and
forming a porous mass by diffusing the biocompatible solvent system from the fluorinated copolymer into the bodily fluids, the porous mass comprising a plurality of filamentous structures that cooperate to define a plurality of macropores having an average diameter greater than 1 μm and accounting for at least 20% void volume of the porous mass, the porous mass separating the adjacent organ structures or tissue structures of the patient.
